(19) United States
(12) Patent Application Publication
(10) Pub. No.: US 2018/0256105 A1
(43) Pub. Date: Sep. 13, 2018

(54) FLEXIBLE WEARABLE DRY ELECTRODE AND PREPARATION METHOD THEREOF

(71) Applicant: Suzhou Mindom Technology Co., Ltd., Changshu, Jiangsu (CN)

(72) Inventors: Xiaohu Gu, Changshu (CN); Youke Huang, Changshu (CN)

(21) Appl. No.: 15/548,404

(22) PCT Filed: Sep. 21, 2016
(86) PCT No.: PCT/CN2016/099601

§ 371 Date: Aug. 2, 2017

(30) Foreign Application Priority Data
Nov. 26, 2015 (CN) 201510836535.7

(51) Int. Cl. A61B 5/00; A61B 5/0408; A61B 5/0478; A61B 5/0492; C09D 5/24; C09D 101/02; C09D 171/02; C09J 183/04; C08J 7/04; C09J 5/00

(52) U.S. Cl. CPC A61B 5/6804; C09J 2467/006; A61B 5/0478; A61B 5/0492; C09D 5/24; C09D 101/02; C09D 171/02; C09J 183/04; C08J 7/047; C09J 5/00; A61B 2562/0215; A61B 2562/125; C08J 2401/02; C08J 2471/02; C08J 2367/02; C09J 2203/326; C09J 2401/001; C09J 2471/001; C09J 2400/16; A61B 5/0408

(57) ABSTRACT

The present invention provides a flexible wearable dry electrode and a preparation method thereof. A flexible wearable dry electrode comprises a base fabric, a transfer glue layer and a nano-conductive layer that are successively attached; the formulation of the transfer glue layer is as follows: weighed in weight percentage, 50%-90% of an elastic resin, 5%-15% of a curing agent, and 5%-35% of a filler; the formulation of the nano-conductive layer is as follows: weighed in parts by weight, 0.1-20 parts of a conductive nanomaterial, 0.1-30 parts of a dispersant, and 0.01-5 parts of a binder. The preparation method thereof is as follows: a conductive coating liquid and a transfer glue are prepared respectively, then both are successively transferred to a flexible release film and finally press-fit onto a base fabric, and subsequently, curing is achieved and the release film can then just be torn off.

### CROSS-REFERENCE TO RELATED APPLICATION(S)

The present application claims priority under 35 U.S.C. §365 to International Patent Application No. PCT/CN2016/099601 filed Sep. 21, 2016, which claims priority to Chinese Patent Application No. 201510836535.7, filed Nov. 26, 2015, each of which are incorporated herein by reference into the present disclosure as if fully set forth herein.

### TECHNICAL FIELD

The present invention relates to the field of flexible electrode materials, and specifically to a flexible wearable dry electrode and a preparation method thereof.

### BACKGROUND

With the social progress and economic development, the mobility and intelligentization of medical equipment have become an irreversible trend, especially the intelligentization, mobility and household use of ECG, EEG and EMG equipment closely related to human health. However, both ECG electrodes, EEG electrodes and EMG electrodes extensively used in clinical medicine, and conductive rubbers applied to heart rate monitors worn by ordinary people during exercise have numerous insuperable problems.

For example, ECG electrodes used in clinical practice are mostly disposable Ag/AgCl electrodes with conductive paste thereon, although such electrodes can provide medical patients with transient reliable ECG signals of patients to be detected, they are discarded after one-time use. Moreover, due to the presence of conductive paste, if the Ag/AgCl electrodes are used for monitoring ECG signals for a long time, the conductive paste would, on the one hand, gradually dehydrated and dried, which would easily cause the detachment of the electrodes and a drastic variation of contact impedance, hereby affecting the stability of the signals; and on the other hand, the presence of conductive paste would cause uncomfortable reactions of partial patients to be detected, such as skin allergy. Furthermore, during the detection of EEG signals, the hair at a specific area of patients to be detected usually need to be shaved and the specific area need to daub the conductive paste, the process of which is cumbersome and would bring numerous inconvenience to the patients to be detected. Furthermore, the electrode used on heart rate tests used for heart rate test of normal people is usually a conductive rubber, such electrodes themselves usually have a relatively poor conductivity, and the contact resistance thereof with human skin is also relatively great, which results in that they usually require water infiltration prior to normal use, and the ECG signals of human during exercise cannot be effectively tested.

Thus, with the improvement of people's living standards and more concerns about their own health, and with the popularization of wearable mobile medical products, it is a development trend of future human body sensing electrodes to organically integrate sensing electrodes with garment materials and give textiles with functionality.

There have been a good number of reports about novel dry, flexible and wearable electrodes, for example, the Chinese patent application No. 201510168806.6 reported a preparation method of a graphene-based flexible electrode; the Chinese patent application No. 201510011794.6 reported a preparation method of an inwrought fluff flexible electrocardiogram electrode. However, the currently existing reports fail to effectively solve the following problems: (1) washing resistance, rubbing resistance and stretching resistance: wearable electrodes have to be able to withstand repeated rinsing and effectively maintain their characteristics, regardless of medical monitoring or motion monitoring; and the electrodes would inevitably be pulled after being placed on garments, which requires that the electrodes have good elasticity and contractibility, and still can effectively maintain their inherent conduction characteristic even after stretching and rebounding; (2) long-time stability: especially in case of being combined with clothing, the wearable type is required to have a relatively long lifetime, to be able to withstand hot iron and direct sunlight etc., and to substantially keep a lifetime equaling to that of the clothing; (3) flexibility and comfortableness: as the part directly contacting human skin, the electrodes need to have sufficient flexibility to reduce the feeling of friction, while to avoid causing problems such as skin allergy, that is to say, the comfortableness is to be assured; and (4) environment tolerance of human body: especially electrodes contacting human bodies need to be able to tolerate the erosion caused by perspiration of human bodies. Just because the existing various types of dry electrodes confront these problems as mentioned above, it is imperative to develop a novel flexible wearable electrode that can overcome the above-mentioned problems.

In view of this, the present invention is specially proposed.

### SUMMARY

A first object of the present invention is to provide a flexible wearable dry electrode, which has the characteristics of high conductivity, good flexibility, high environmental tolerance, long-time stability, washing resistance and rubbing resistance, and can be discretionarily cut out according to the requirements of garments and accordingly has a high universality.

A second object of the present invention is to provide a preparation method of the flexible wearable dry electrode, wherein a traditional coating mode is introduced into the preparation method, thus, the production costs can be effectively reduced and mass production can be achieved.

In order to achieve the above-mentioned objects of the present invention, the following technical solutions are specifically adopted:

A flexible wearable dry electrode, comprising a base fabric, a transfer glue layer and a nano-conductive layer that are successively attached; the transfer glue layer is mainly composed of the following components: weighed in weight percentage, 50%-90% of an elastic resin, such as 55%, 60%, 65%, 70%, 75%, 80%, and 85%; 5%-15% of a curing agent, such as 6%, 7%, 8%, 9%, 10%, 11%, 12%, 13%, and 14%; and 5%-35% of a filler, such as 10%, 15%, 20%, 25%, and 30%; the nano-conductive layer is mainly composed of the following components: weighed in parts by weight, 0.1-20 parts of a conductive nanomaterial, such as 0.5 part, 1 part, 5 parts, 10 parts, and 15 parts; 0.1-30 parts of a dispersant, such as 0.5 part, 1 part, 5 parts, 10 parts, 15 parts, 20 parts, and 25 parts; and 0.01-5 parts of a binder, such as 0.05 part, 0.1 part, 0.5 part, 1 part, 2 parts, 3 parts, and 4 parts.

Compared with a traditional flexible electrode, firstly, as to the above-mentioned flexible wearable dry electrode of the present invention, a transfer glue layer is added between the conductive layer and the fabric, wherein the conductive layer is stably adhered to the base fabric by taking the transfer glue layer as adhering bridge, hereby solving the problem of lack of washing resistance, rubbing resistance, and stretching resistance.

Secondly, the formulas and the formulation ratios for the transfer glue layer and the nano-conductive layer are respectively improved in the present invention, such that the transfer glue layer and the nano-conductive layer have a better fusion effect and are improved in the aspects of flexibility, long-time stability, and environmental tolerance; for example, in the nano-conductive layer, the dispersant is used to sufficiently disperse the conductive nanomaterial in the solvent, such that the conductive material is uniformly distributed on the respective areas of the fabric; and the binder is used to adhere free molecules in the dispersed solution together, hereby functioning as a framework, such that the whole nano-conductive solution can have a suitable mobility for forming a laminated shape, rather than not being able to form a shape due to being too dispersed; whereas in the transfer glue layer, an elastic resin is selected, in order to assure that the product has a good tensile strength for avoiding influence on the conductivity due to the tension; and a curing agent is regularly used for the purpose of curing; while the filler is used to enhance the mechanical strength of the transfer glue layer, such that the fabric still has an excellent mechanical strength even after the attachment of the conductive material, and has a functional performance same as that of an ordinary fabric.

Thirdly, the present invention has the characteristic of a multilayer structure, which not only solves the problem regarding the adhesion and flexibility of the three different kinds of materials, and can satisfy the requirement of motion process monitoring; but also completely exposes the nano-conductive layer to the outside, and the impedance is reduced, such that the final products have a relatively high conductivity, and thereby an electrical signal from a human body can be effectively and accurately detected.

Furthermore, when being worn, the flexible wearable dry electrode in the present invention directly contacts the human skin via the nano-conductive layer, while the materials of general nano-conductive layers are relatively safe. Thus, the biological safety of the product of the present invention is relatively high.

Thus, it can be seen that the present invention not only solves the problems that commonly exist regarding the existing dry flexible wearable electrodes, but also improves indices such as environmental tolerance and conductivity. Thus, comparatively speaking, the present invention has more prominent advantages and a wider application prospect.

The above-mentioned flexible wearable dry electrode may further be improved as follows:

Preferably, the conductive nanomaterial is one or a mixture of several from copper nanosheet, copper nanowire, silver nanowire, silver nanosheet, silver nanoparticle, gold nanowire, gold nanosheet, platinum nanowire, palladium nanowire, palladium nanosheet, bismuth nanowire, bismuth nanosheet, nickel nanowire, nickel nanosheet, cobalt nanowire, cobalt nanosheet, gold-silver alloy nanowire, gold-silver alloy nanotube, platinum-silver alloy nanotube, platinum-palladium alloy nanowire, carbon nanotube, carbon nanofiber, graphene, indium tin oxide (ITO) nanowire and the like. Actually, the present invention may adopt all kinds of conductive base materials, for example, traditional metallic conductive materials or novel conductive polymeric materials. The above-mentioned conductive nanomaterials are preferably adopted, as these conductive materials have a high compatibility with other additives, and have a macroscopical shape of powders and are easy to mix and dissolve.

Preferably, the dispersant is one or a mixture of several from polyethylene glycol, polyvinyl alcohol, polyvinyl pyrrolidone, hydroxypropyl methyl cellulose, hexadecyl trimethyl ammonium bromide, sodium laurate, sodium cinnamate, sodium oleate, sodium dodecyl benzenesulfonate, sodium dodecyl sulfonate, carboxymethyl cellulose acetate butyrate, carboxymethyl cellulose, Acacia gum, sodium citrate, styrene-maleic anhydride copolymer, aqueous polyurethane, and aqueous epoxy resin. These dispersants can better facilitate the sufficient suspension and dispersion of the conductive nanomaterial in the solvent, and maintain a good stable state.

Preferably, the binder is one or a mixture of several from isocyanate, polyamide, modified aliphatic amine, aromatic polyamine, maleic anhydride, and urea. These binders can assure that a nano-conductive fluid forming the nano-conductive layer is firmly fixed on the base fabric in the form of a layer, rather than in a mode of impregnation and infiltration, hereby assuring that the material has sufficient conductivity and flexibility.

Preferably, the elastic resin is one or a mixture of several from acrylic resin, polyurethane, modified silica gel resin, and modified epoxy resin. These resins can assure that the formed electrode has sufficient elasticity, and allow the electrode to be rapidly restored from stretching and to have inherent conductivity characteristics.

Preferably, the curing agent is one or a mixture of several from platinum catalyst, amino resin, isocyanate, polyamide, modified aliphatic amine, aromatic polyamine, maleic anhydride, urea, phenolic resin, and dicyanodiamide. The curing conditions of these curing agents are milder and relatively easy to realize.

Preferably, the filler is one or a mixture of several from silicon dioxide powder, fumed silicon dioxide powder, titanium dioxide, activated carbon, calcium carbonate, carbon black, α-cellulose, mica, zinc oxide, and calcium silicate. The mechanical strength of the transfer glue is higher after the use of these fillers.

The preparation method of the flexible wearable dry electrode as mentioned above includes the steps of:
adding a solvent to all raw materials for mixing and dissolving according to a formulation of the nano-conductive layer to provide a conductive coating liquid;
mixing and dissolving all raw materials according to a formulation of the transfer glue layer to provide a transfer glue;
coating a flexible release film with the conductive coating liquid with a coating thickness ranging between 5 and 500 microns to provide a flexible conductive carrier available for transfer printing;
coating a conductive plane side of the flexible conductive carrier with the transfer glue with a coating thickness ranging between 50 and 2000 microns to provide a conductive-glue composite carrier; and
press-fitting the conductive-glue composite carrier on the base fabric in a way of exposing the flexible release film to outside, and then heating for curing, and finally tearing off the flexible release film.

In the above-mentioned preparation method, the conductive coating liquid and the transfer glue are respectively prepared, and then successively coated on a flexible release film, which serves as calibration plate, hereby forming a composite carrier, and finally, the composite carrier is press-fit with the base fabric, and the flexible release film is torn off after the curing of the transfer glue and the conductive coating liquid, which leads to the product. Accordingly, the traditional coating mode is introduced into the above-mentioned preparation method, which can effectively reduce the production costs and can realize mass production; moreover, with respect to other production modes (pre-impregnation etc.), the quality is more controllable, the production efficiency is higher, and the resulting material also has a stronger performance.

The above-mentioned preparation method can be improved as follows:

Preferably, the method of press-fitting is achieved by: applying a pressure of 0.001 MPa-5 MPa (e.g. 0.005 MPa, 0.01 MPa, 0.05 MPa, 0.1 MPa, 0.5 MPa, 1 MPa, 2 MPa, 3 MPa, and 4 MPa), and keeping this pressure for 5-300 seconds (e.g. 10 seconds, 50 seconds, 100 seconds, 150 seconds, 200 seconds, and 250 seconds). The relationship between pressure and time is balanced via this method, that is to say, consideration is given to both the operation difficulty level and the production cycle, and the cost performance is high.

Preferably, the method of heat curing is achieved by: performing baking for 5-120 minutes (e.g. 10 minutes, 30 minutes, 50 minutes, 70 minutes, 90 minutes, and 110 minutes) at a temperature of 80-300° C. (e.g. 100° C., 150° C., 200° C., and 250° C.). This temperature allows both the curing, and complete evaporation of the solvent in the conductive fluid. Furthermore, during baking, baking at a constant temperature is optimal, and the material obtained so has a more stable quality.

Furthermore, it is sufficient that the flexible release film employed in the preparation method can realize the function of release, for example, it may be one selected from polyethylene terephthalate (PET) thin film, polycarbonate (PC) thin film, polyvinyl chloride (PVC) thin film, polyethylene (PE) thin film, polypropylene (PP) thin film, polyurethane (PU) thin film, silica gel thin film, polyvinyl alcohol (PVA) thin film, polytetrafluoroethylene thin film, polyvinylidene fluoride thin film and the like.

Preferably, the solvent is one or a mixture of several from water, ethanol, isopropanol, ethylene glycol, glycerol, isophorone, DBE, dichloroethane, trichloroethane, toluene, xylene, 1,4-dioxane, propylene glycol methyl ether, propylene glycol ethyl ether, carbitol acetate, carbitol caproate, diacetone alcohol, and diacetone. As these solvents have a relatively high solubility or dispersibility towards the conductive nanomaterials, and are volatile, so they are easy to dry and remove during the preparation process.

Compared with the prior art, the present invention has the following beneficial effects:

(1) The product has the characteristics of high conductivity, good flexibility, high environmental tolerance, long-time stability, washing resistance and rubbing resistance, and wide application range and the like. Specifically, the final product may have a surface resistance value in the range of 0.01 mΩ/□ and 50Ω/□, be bent with a discretionary curvature, completely equal the flexibility of the fabric material, and can resist the erosion caused by acid, alkaline, and salt constitution of human bodies, that is to say, the structural characteristics and conductive characteristics of the electrode can be effectively maintained in a 0.1-10% sodium chloride aqueous solution having a pH value of 6-9, and sunlight exposure could be withstood without causing the increase of resistance; ironing at 100-200° C. for 10-120 minutes does not cause any deformation of the electrode and any variation in the resistance; the placement under a temperature of −10-200° C. and a humidity of 20%-100% for over 12-24 months causes no variation; washing for 2-48 hours at a water temperature of 0-80° C. with various types of detergents causes no variation in the structure and resistance of the electrode; there is no variation in the electrode conductivity and in the electrode appearance after relative rubbing of the conductive planes for 100-400 times; and all kinds of base fabrics (cotton, fiber, polyester and the like) can be adopted.

(2) It can be prepared into a discretionary shape and can be discretionarily trimmed, which means a high machinability.

(3) The preparation method is simple and efficient, and the process can be well controlled, and mass production is easy to realize.

### BRIEF DESCRIPTION OF THE DRAWING

In order to more clearly explain the examples of the present invention or the technical solutions in the prior art, below the drawing to be used in the description of the examples or the prior art will be briefly introduced.

FIG. 1 is a schematic flow chart for the preparation of a flexible wearable dry electrode provided in example 1 of the present invention.

### DETAILED DESCRIPTION

In the following contents, the embodiments of the present invention will be described in detail with reference to the examples; however, a person skilled in the art would understand that the following examples are merely used to explain the present invention, rather than being deemed as limiting the scope of the present invention. Examples, for which no concrete situations are specified, are performed according to conventional situations or situations recommended by the manufactures. Reagents or instruments, for which no manufacturers are specified, are conventional products available commercially.

### Example 1

A flexible wearable dry electrode:

Step (1): a nano-conductive coating liquid was firstly prepared, wherein 1 g of copper nanowire, 1 g of silver nanowire and 1 g of graphene were taken and simultaneously dispersed into 25 g of mixed solvent included water, ethanol and ethylene glycol, and then 2 g of polyethylene glycol and 0.5 g of hydroxypropyl methyl cellulose (molecular weight of 20000) were added, and after thorough stirring of and uniform dissolution, 0.2 g of curing agent comprising isocyanate, was added and the solution was continuously stirred till it was completely uniform and then for standby use.

Step (2): a transfer glue was prepared, wherein 5 g of acrylic ester (Quanzhi Shanghai, type R20B) and 3 g of modified silica gel resin (Shanghai Resin Factory Co., LTD., type 665) were taken and mixed uniformly, then 1.5 g of gas-phased silicon dioxide and 0.5 g of titanium dioxide were added and thoroughly stirred uniformly, and 0.1 g of platinum catalyst and 0.3 g of isocyanate were subsequently added, the obtained substance was continuously stirred uniformly for standby use.

Step (3): the nano-conductive coating liquid obtained in step (1) was coated onto a PET thin film substrate via a coating machine and with a coating thickness of 150 microns, which leads to a conductive carrier with an attached nano-conductive layer, the surface resistance of the conductive carrier was 200 mΩ/□.

Step (4): the transfer glue obtained in step (2) was coated on the conductive plane of the conductive carrier obtained in step (3) with a coating thickness of 200 microns, which leads to a conductive-glue composite carrier.

Step (5): the glue plane of the conductive-glue composite carrier obtained in step (4) was attached to the fabric, and the two were press-fit for 30 seconds with a pressure of 0.5 MPa, and the product was subsequently placed in a dry oven at a temperature of 150° C. for baking for 20 minutes. After the baking, the product was taken out and the PET substrate was torn off, which leads to a fabric-based flexible electrode.

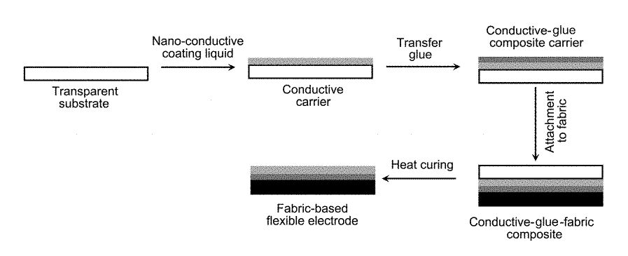

The flow path of the above-mentioned preparation method is shown in FIG. 1.

Performance test: The surface resistance of the electrode was 200 mΩ/□. The resulting flexible electrode also showed flexibility and elasticity that completely match ordinary clothes and could realize a relatively good contact with human skin, and no variation on the electrode resistance occurred after soaking in a 5% sodium chloride aqueous solution having a pH value of 8.5 for 60 minutes; after being insolated under the sunlight for 8 hours, the electrode resistance did not change, and no variation occurred regarding the physical properties thereof; there was no electrode deformation and no resistance variation after steam ironing at 200° C. for 30 minutes; the product could be placed under a temperature of 100° C. and a humidity of 90% for over 12 months without any variation; washing for 10 hours at a water temperature of 60° C. with various types of detergents caused no variation in electrode structure and in electrode resistance; and there was no variation in the electrode conductivity and in the electrode appearance after relative rubbing of the conductive planes for 200 times. The content mentioned above shows that the flexible electrode prepared via the coating-transfer mode has good functional characteristics.

### Example 2

A flexible wearable dry electrode:

Step (1): a nano-conductive coating liquid was firstly prepared, wherein 2 g of silver nanowire, 1 g of carbon nanotube and 1 g of graphene were taken and simultaneously dispersed into 30 g of mixed solvent containing water, ethylene glycol and isophorone, and then 1 g of polyvinyl alcohol (molecular weight of 40000), 0.5 g of hydroxypropyl methyl cellulose (molecular weight of 400) and 1 g of sodium laurate were added, and after thorough stirring and uniform dissolution, curing agents comprising 0.2 g of isocyanate and 0.1 g of urea, were added and the solution was continuously stirred till it was completely uniform and then for standby use.

Step (2): a transfer glue was prepared, wherein 20 g of modified silica gel resin (Shanghai Resin Factory Co., LTD., type 665) was taken and mixed uniformly, then 1 g of gas-phased silicon dioxide and 1.5 g of titanium dioxide were added and thoroughly stirred uniformly, and 0.5 g of platinum catalyst and 1 g of isocyanate were subsequently added, the obtained substance was continuously stirred uniformly for standby use.

Step (3): the nano-conductive coating liquid obtained in step (1) was coated onto a PC thin film substrate via a coating machine and with a coating thickness of 120 microns, which leads to a conductive carrier with an attached nano-conductive layer, the surface resistance of the conductive carrier was 80 mΩ/□.

Step (4): the transfer glue obtained in step (2) was coated on the conductive plane of the conductive carrier obtained in step (3) with a coating thickness of 250 microns, which leads to a conductive-glue composite carrier.

Step (5): the glue plane of the conductive-glue composite carrier obtained in step (4) was attached to the fabric, and the two were press-fit for 60 seconds with a pressure of 0.8 MPa, and the product was subsequently placed in a dry oven at a temperature of 80° C. for baking for 100 minutes. After the baking, the product was taken out and the PC substrate was torn off, which leads to a fabric-based flexible electrode.

Performance test: The surface resistance of the electrode was still 80 mΩ/□, and the conductive characteristics of the original conductive carrier were effectively maintained. The resulting flexible electrode also showed flexibility and elasticity that completely match ordinary clothes and could realize a relatively good contact with human skin, and no variation on the electrode resistance occurred after soaking in a 8% sodium chloride aqueous solution having a pH value of 7.5 for 80 minutes; after being insolated under the sunlight for 24 hours, the electrode resistance did not change, and no variation occurred regarding the physical properties thereof; there was no electrode deformation and no resistance variation after steam ironing at 180° C. for 60 minutes; the product could be placed under a temperature of 180° C. and a humidity of 80% for 15 months without any variation; washing for 24 hours at a water temperature of 80° C. with various types of detergents caused no variation in electrode structure and in electrode resistance; and there was no variation in the electrode conductivity and in the electrode appearance after relative rubbing of the conductive planes for 300 times. The content mentioned above shows that the flexible electrode prepared via the coating-transfer mode has good functional characteristics.

### Example 3

A flexible wearable dry electrode:

Step (1): a nano-conductive coating liquid was firstly prepared, wherein 0.05 g of copper nanowire, 0.1 g of silver nanowire and 0.2 g of graphene were taken and simultaneously dispersed into 300 g of mixed solvent containing water, ethylene glycol and glycerol, and then 0.1 g of polyvinyl alcohol (molecular weight of 20000), 0.15 g of hydroxypropyl methyl cellulose (molecular weight of 40000) and 0.1 g of sodium laurate were added, and after thorough stirring and uniform dissolution, binder comprising 0.02 g of isocyanate and 0.02 g of urea, were added and the solution was continuously stirred till it was completely uniform and then for standby use.

Step (2): a transfer glue was prepared, wherein 45 g of modified silica gel resin (Shanghai Resin Factory Co., LTD., type 665) was taken and mixed uniformly, then 1 g of gas-phased silicon dioxide and 1.5 g of titanium dioxide were added and thoroughly stirred uniformly, and 2.5 g of platinum catalyst was subsequently added, the obtained substance was continuously stirred uniformly for standby use.

Step (3): the nano-conductive coating liquid obtained in step (1) was coated onto a PC thin film substrate via a coating machine and with a coating thickness of 25 microns, which leads to a conductive carrier with an attached nano-conductive layer, the surface resistance of the conductive carrier was 50 mΩ/□.

Step (4): the transfer glue obtained in step (2) was coated on the conductive plane of the conductive carrier obtained in step (3) with a coating thickness of 800 microns, which leads to a conductive-glue composite carrier.

Step (5): the glue plane of the conductive-glue composite carrier obtained in step (4) was attached to the fabric, and the two were press-fit for 300 seconds with a pressure of 5 MPa, and the product was subsequently placed in a dry oven at a temperature of 80° C. for baking for 120 minutes. After the baking, the product was taken out and the PC substrate was torn off, which leads to a fabric-based flexible electrode. Wherein the surface resistance of the electrode was still 50 mΩ/□, and the conductive characteristics of the original conductive carrier were effectively maintained. The resulting flexible electrode also showed flexibility and elasticity that completely match ordinary clothes and could realize a relatively good contact with human skin, and no variation on the electrode resistance occurred after soaking in a 0.1% sodium chloride aqueous solution having a pH value of 6 for 120 minutes; after being insolated under the sunlight for 48 hours, the electrode resistance did not change, and no variation occurred regarding the physical properties thereof; there was no electrode deformation and no resistance variation after steam ironing at 200° C. for 120 minutes; the product could be placed under a temperature of 200° C. and a humidity of 100% for 24 months without any variation; washing for 48 hours at a water temperature of 0° C. with various types of detergents caused no variation in electrode structure and in electrode resistance; and there was no variation in the electrode conductivity and in the electrode appearance after relative rubbing of the conductive planes for 400 times. The content mentioned above shows that the flexible electrode prepared via the coating-transfer mode has good functional characteristics.

### Example 4

A flexible wearable dry electrode:

Step (1): a nano-conductive coating liquid was firstly prepared, wherein 2 g of copper nanowire, 6 g of silver nanowire and 2 g of carbon nanotube were taken and simultaneously dispersed into 22.5 g of mixed solvent containing water, ethanol and glycerol, and then 10 g of polyvinyl alcohol (molecular weight of 40000), 2.5 g of carboxymethyl cellulose acetate butyrate (CMCAB-641-0.2) and 2.5 g of sodium cinnamate were added, and after thorough stirring and uniform dissolution, binder comprising 1.25 g of isocyanate and 1.25 g of urea, were added and the solution was continuously stirred till it was completely uniform and then for standby use.

Step (2): a transfer glue was prepared, wherein 25 g of modified silica gel resin (Shanghai Resin Factory Co., LTD., type 665) was taken and mixed uniformly, then 17.5 g of titanium dioxide was added and thoroughly stirred uniformly, and 7.5 g of platinum catalyst was subsequently added, the obtained substance was continuously stirred uniformly for standby use.

Step (3): the nano-conductive coating liquid obtained in step (1) was coated onto a PET thin film substrate via a coating machine and with a coating thickness of 350 microns, which leads to a conductive carrier with an attached nano-conductive layer, the surface resistance of the conductive carrier was 0.01 mΩ/□.

Step (4): the transfer glue obtained in step (2) was coated on the conductive plane of the conductive carrier obtained in step (3) with a coating thickness of 1000 microns, which leads to a conductive-glue composite carrier.

Step (5): the glue plane of the conductive-glue composite carrier obtained in step (4) was attached to the fabric, and the two were press-fit for 5 seconds with a pressure of 0.001 MPa, and the product was subsequently placed in a dry oven at a temperature of 300° C. for baking for 5 minutes. After the baking, the product was taken out and the PC substrate was torn off, which leads to a fabric-based flexible electrode. Wherein the surface resistance of the electrode was still 0.01 mΩ/□, and the conductive characteristics of the original conductive carrier were effectively maintained. The resulting flexible electrode also showed flexibility and elasticity that completely match ordinary clothes and could realize a relatively good contact with human skin, and no variation on the electrode resistance occurred after soaking in a 10% sodium chloride aqueous solution having a pH value of 9 for 300 minutes; after being insolated under the sunlight for 24 hours, the electrode resistance did not change, and no variation occurred regarding the physical properties thereof; there was no electrode deformation and no resistance variation after steam ironing at 100° C. for 10 minutes; the product could be placed under a temperature of 100° C. and a humidity of 20% for 24 months without any variation; washing for 12 hours at a water temperature of 80° C. with various types of detergents caused no variation in electrode structure and in electrode resistance; and there was no variation in the electrode conductivity and in the electrode appearance after relative rubbing of the conductive planes for 200 times. The content mentioned above shows that the flexible electrode prepared via the coating-transfer mode has good functional characteristics.

### Comparative Example

A traditional flexible electrode, prepared in the following mode: after the mixture of conductive carbon powders with a silicone rubber or a polyurethane resin, the obtained mixture was shaped by pouring into a mold and the heat curing.

Performance test: The surface resistance of the electrode was 500 mΩ/□; as the conductive material was mixed with a resin and a relatively good flexibility had to be maintained, the original conductive characteristics of the conductive material could not be maintained; due to the natural incompatibility of the conductive carbon powders with the resin material that is mixed therewith, so that the resulting flexible electrode could not be totally matched to the flexibility and elasticity of ordinary clothing fabrics, and the contact of the flexible electrode with the human skin was just acceptable, however, the contact impedance was great; no variation on the electrode resistance occurred after soaking in a 0.1% sodium chloride aqueous solution having a pH value of 6 for 60 minutes; after being insolated under the sunlight for 8 hours, the electrode hardened and the resistance increased; after steam ironing at 100° C. for 10 minutes, the electrode deformed while no significant variation in resistance occurred; the electrode deformed and was embrittled, and the resistance increased exponentially after placement under a temperature of 100° C. and a humidity of 90% for 12 months; after washing for 10 hours at a water temperature of 60° C. with various types of detergents, the electrode was broken, while no significant variation in resistance occurred; and no significant variation in the electrode conductivity occurred, and the electrode appearance was significantly roughened after relative rubbing of the conductive planes for 200 times. The content mentioned above shows that the functional characteristics of the traditional flexible electrode are relatively poor.

Although the present invention has already been explained and described through specific examples, it shall be aware that many further modifications and variations may also be made without departing from the spirit and scope of the present invention. Thus, it means that all these modifications and variations falling in the scope of the present invention are included in the appended claims.

1. A flexible wearable dry electrode, comprising a base fabric, a transfer glue layer and a nano-conductive layer, which are successively attached;
wherein the transfer glue layer is mainly composed of the following components, by weight percentage: 50%˜90% of an elastic resin, 5%˜15% of a curing agent, and 5%˜35% of a filler; and
the nano-conductive layer is mainly composed of the following components, in parts by weight: 0.1˜20 parts of a conductive nanomaterial, 0.1˜30 parts of a dispersant, and 0.01˜5 parts of a binder.

2. The flexible wearable dry electrode according to claim 1, wherein the dispersant is one selected from the group consisting of polyethylene glycol, polyvinyl alcohol, polyvinyl pyrrolidone, hydroxypropyl methyl cellulose, hexadecyl trimethyl ammonium bromide, sodium laurate, sodium cinnamate, sodium oleate, sodium dodecyl benzenesulfonate, sodium dodecyl sulfonate, carboxymethyl cellulose acetate butyrate, carboxymethyl cellulose, Acacia gum, sodium citrate, styrene-maleic anhydride copolymer, aqueous polyurethane, and aqueous epoxy resin or a mixture of several of the group.

3. The flexible wearable dry electrode according to claim 1, wherein the dispersant is one selected from the group consisting of polyethylene glycol, hydroxypropyl methyl cellulose, polyvinyl alcohol, and sodium laurate or a mixture of several of the group.

4. The flexible wearable dry electrode according to claim 1, wherein the binder is one selected from the group consisting of isocyanate, polyamide, modified aliphatic amine, aromatic polyamine, maleic anhydride, and urea or a mixture of several of the group.

5. The flexible wearable dry electrode according to claim 1, wherein the binder is embodied as isocyanate and/or urea.

6. The flexible wearable dry electrode according to claim 1, wherein the elastic resin is one selected from the group consisting of acrylic resin, polyurethane, modified silica gel resin, and modified epoxy resin or a mixture of several of the group.

7. The flexible wearable dry electrode according to claim 1, wherein the elastic resin is embodied as modified silica gel resin and/or acrylic resin.

8. The flexible wearable dry electrode according to claim 1, wherein the curing agent is one selected from the group consisting of platinum catalyst, amino resin, isocyanate, polyamide, modified aliphatic amine, aromatic polyamine, maleic anhydride, urea, phenolic resin, and dicyanodiamide or a mixture of several of the group.

9. The flexible wearable dry electrode according to claim 1, wherein the curing agent is embodied as platinum catalyst and/or isocyanate.

10. The flexible wearable dry electrode according to claim 1, wherein the filler is one selected from the group consisting of silicon dioxide powder, fumed silicon dioxide powder, titanium dioxide, activated carbon, calcium carbonate, carbon black, α-cellulose, mica, zinc oxide, and calcium silicate or a mixture of several of the group.

11. The flexible wearable dry electrode according to claim 1, wherein the filler is embodied as fumed silicon dioxide powder and/or titanium dioxide.

12. The flexible wearable dry electrode according to claim 1, wherein the conductive nanomaterial is one selected from the group consisting of copper nanosheet, copper nanowire, silver nanowire, silver nanosheet, silver nanoparticle, gold nanowire, gold nanosheet, platinum nanowire, palladium nanowire, palladium nanosheet, bismuth nanowire, bismuth nanosheet, nickel nanowire, nickel nanosheet, cobalt nanowire, cobalt nanosheet, gold-silver alloy nanowire, gold-silver alloy nanotube, platinum-silver alloy nanotube, platinum-palladium alloy nanowire, carbon nanotube, carbon nanofiber, graphene, and indium tin oxide nanowire or a mixture of several of the group.

13. The flexible wearable dry electrode according to claim 1, wherein the conductive nanomaterial is one selected from the group consisting of copper nanowire, silver nanowire, carbon nanotube, and graphene or a mixture of several of the group.

14. A method for preparing the flexible wearable dry electrode according to any of claims 1, comprising steps of:
adding a solvent to all raw materials for mixing and dissolving according to a formulation of the nano-conductive layer to provide a conductive coating liquid;
mixing and dissolving all raw materials according to a formulation of the transfer glue layer to provide a transfer glue;
coating a flexible release film with the conductive coating liquid to provide a flexible conductive carrier available for transfer printing;
coating a conductive plane side of the flexible conductive carrier with the transfer glue to provide a conductive-glue composite carrier; and
press-fitting the conductive-glue composite carrier on the base fabric in a way of exposing the flexible release film to outside, and then heating for curing, and finally tearing off the flexible release film.

15. The method according to claim 14, wherein the press-fitting is achieved by a method of: applying a pressure of 0.001 MPa˜5 MPa and keeping the pressure for 5˜300 seconds.

16. The method according to claim 14, wherein heat curing is achieved by a method of: baking at 80˜300° C. for 5˜120 minutes.

17. The method according to claim 16, wherein the baking is achieved at a constant temperature.

18. The method according to claim 14, wherein the solvent is one selected from the group consisting of water, ethanol, isopropanol, ethylene glycol, glycerol, isophorone, DBE, dichloroethane, trichloroethane, toluene, xylene, 1,4-dioxane, propylene glycol methyl ether, propylene glycol ethyl ether, carbitol acetate, carbitol caproate, diacetone diacetone alcohol, and diacetone or a mixture of several of the group.

19. The method according to claim 14, wherein the solvent is one selected from the group consisting of water, ethanol, isophorone and ethylene glycol or a mixture of several of the group.

20. The method according to claim 14, wherein the flexible release film is selected from one of polyethylene terephthalate thin film, polycarbonate thin film, polyvinyl chloride thin film, polyethylene thin film, polypropylene thin film, polyurethane thin film, silica gel thin film, polyvinyl alcohol thin film, polytetrafluoroethylene thin film, and polyvinylidene fluoride thin film.
